(19) United States
(12) Patent Application Publication
(10) Pub. No.: US 2013/0317622 A1
(43) Pub. Date: Nov. 28, 2013

(54) SURGICAL METHOD FOR GRAFTING AN ARTIFICIAL IMPLANT IN A BLADDER AND/OR IN A URETHRAL OR URETERAL SEGMENT

(71) Applicants: Antonio SAMBUSSETI, Cremona (IT); Gianni CANCARINI, Brescia (IT)

(72) Inventors: Antonio SAMBUSSETI, Cremona (IT); Gianni CANCARINI, Brescia (IT)

(73) Assignee: Antonio SAMBUSSETI, Cremona (IT)

(21) Appl. No.: 13/481,135

(22) Filed: May 25, 2012

(51) Int. Cl. A61F 2/04

(52) U.S. Cl. 623/23.65

(57) ABSTRACT

A description is given of a method for implanting a prosthesis or artificial implant (100) to a bladder and/or to a urethral/ureteral segment in the surgical treatment of diseases of the bladder and/or of the urethra/ureters by removal of a diseased portion of bladder and/or of urethra/ureters, characterised in that the edge of the prosthesis or artificial implant (100) is interposed between the muscle tissue (5) and the underlying portion of urothelial tissue (6) at the removed bladder portion or at the free ends of the urethra/ureter portions (segments) remaining after removal of the diseased segment, so as to be sandwich sutured between the muscle tissue and the urothelial tissue.

The present invention relates to a method for the grafting of a prosthesis (or artificial implant) in a bladder and/or in a ureteral or urethral segment in the surgical treatment of diseases of the bladder and of the urethra/ureters.

More particularly the present invention relates to a surgical method for the grafting of an artificial patch for the replacement of a portion of bladder wall following bilharzia, and/or of an artificial cuff for bladder augmentation, and/or of an artificial tubular device for the reconstruction of a urethral or ureteral segment.

As is known, when the bladder or the urethra/ureters of a patient is affected by a serious disease the portion of bladder or of urethra/ureters affected must be removed to prevent the disease extending to the remaining part of bladder or of urethra/ureters.

In the case of the bladder, the removal of this portion of bladder wall creates a hole which is closed by means of the application of a patch which is sutured around the perimeter of this hole.

The patches used for this application can be taken from the inverted intestine of the patient (autologous patches) or be specially made from artificial materials, resorbable or non-resorbable, such as those described, for example, in the application PCT/EP2006/009274 in the name of the Applicant, which are sutured using the same procedure of grafting of the autologous patches.

The advantage of artificial patches compared to autologous patches lies in the fact that they reduce the onset of infections due to the presence of residues of intestinal mucus on the surface of the autologous patch and the occurrence of phenomena of tearing due to the short average life of the autologous patch taken from the intestinal wall.

Similarly to the artificial patches, artificial tubular devices also exist, suitable for the replacement of a urethral and/or ureteral segment, which are sutured around the remaining segments of the urethra or ureter by end-to-end suturing.

The same can be repeated for bladder augmentation wherein cuff-shaped or hemispherical devices are used, in artificial material, suitable for increasing the overall volume of the bladder with low capacity (compliance), which are also sutured around the opening created on the bladder during the augmentation operation.

Since the tissue of the bladder, of the urethra and of the ureters comprises an inner layer of urothelial tissue (transitional epithelium) whereto numerous functions are attributable including mainly the impermeability to urine, potentially toxic for the organism, as well as the contraction or the relaxation of these organs, it is evident that the grafting of a patch and/or of the other artificial devices mentioned above must be such as to ensure the growth of a bladder or urethra/ureter neotissue that comprises a continuous layer of urothelial tissue (transitional epithelium), which is normally present as lining over the entire inner surface of a healthy bladder and of healthy urethra and ureters.

The object of the present invention is therefore to provide a surgical method for grafting a prosthesis or artificial implant in a bladder and/or in a urethral or ureteral segment in the surgical treatment of diseases of the bladder and/or the urethra/ureters, which enables the urothelial tissue, surrounding the removed part of bladder or urethra/ureter, to re-grow so as to form a continuous layer of urothelial tissue, also overcoming the disadvantages of the prior art.

Another object is that of providing such a method that is suitable for grafting an artificial patch in a bladder in the surgical treatment of bilharzia by means of replacement of a portion of bladder wall, and/or for grafting an artificial cuff to a bladder in the surgical treatment of bladders with low compliance by means of bladder augmentation, and/or for grafting an artificial tubular device to a urethral or ureteral segment for the reconstruction of a urethral or ureteral segment.

A further object is that of providing such a surgical method of grafting which is easy and simple to perform, and also fast.

These objects are achieved by the method of grafting according to the invention having the features listed in the appended independent claim 1.

Advantageous embodiments of the invention are disclosed by the dependent claims.

The object of the present invention is a method for implanting at least one artificial implant (prosthesis) in a bladder and/or in a urethral/ureteral segment in the surgical treatment of diseases of the bladder and/or of the urethra/ureters, which provides for the detaching one from the other of the muscle tissue and the urothelial tissue at the edge of the removed portion of bladder or at the free ends of the portions (segments) of urethra/ureter remaining following removal of a segment affected by disease, and inserting the perimeter edge of the artificial implant between the muscle tissue and the underlying urothelial tissue at the portion of bladder removed or at the free ends of the portions (segments) of the urethra/ureter remaining after removal of said segment affected by disease, so as to sandwich suture said implant between the two muscle and urothelial tissues detached.

In particular the present method comprises the following steps: removing the portion of bladder and/or urethra/ureters segment of a patient affected by a disease;

detaching a portion of muscle tissue from the underlying portion of urothelial tissue at the edge of the above said removed bladder portion and/or at the free ends of the urethra/ureter portions (segments) remaining following the removal of said diseased segment, so as to form detached flaps of muscle and urothelial tissue;

inserting the perimeter edge of an artificial implant selected from among patch, scaffold, cuff, hemispherical device and/or tubular device, between the detached flaps of the two muscle and urothelial tissues;

bringing the previously detached flaps of the two muscle and urothelial tissues together until closing as a sandwich the edge of said patch, scaffold, cuff, hemispherical device and/or tubular device;

passing a suture thread through said flaps of muscle and urothelial tissue brought together so as to sandwich suture the perimeter edge of said patch, scaffold, cuff, hemispherical device and/or tubular device to both said tissues.

Thanks to the fact that the present method provides for the interposition of the perimeter edge of the implant chosen (patch, scaffold, cuff, hemispherical device and/or tubular device) between the muscle tissue and the urothelial tissue, previously separated one from the other at the perimeter of the area/segment removed, it is possible to obtain a homogeneous and uniform growth of the muscle tissue on the upper surface of the implant, and of the urothelial tissue on the lower surface of said implant.

The patch which can be used in this method can be any patch known in the art, absorbable (resorbable) or non-absorbable (non-resorbable), preferably made with a flexible and elastic material.

As an example mention can be made of the patch in soft and elastic silicone completely coated by a thin film made of pyrolytic turbostratic carbon as described in the applications PCT/EP2006/009274, PCT/EP2008/006352 herein incorporated integrally as reference, or made in silicone coated with a thin film of diamond-like carbon as described in the application MI2011A000495 herein incorporated integrally as reference.

In a preferred embodiment the coating of the patch is made of pyrolytic turbostratic carbon.

It is also possible to use in the present method resorbable patches such as those described in patent applications WO2011/064110, MI2012A 000 381 (made in PGA) and in the application MI2009A 000 053 (made in PLA), all in the name of the Applicant.

The thickness of the patch which can be used in the present method is similar to that of the patches already used in the art, for example around 600 microns, while the thickness of the coating thin film is preferably approximately 0.2 to 0.3 micron.

The tubular device which can be used in the present method is also preferably made in elastic and soft silicone coated with pyrolytic turbostratic carbon.

The thickness of the tubular device can vary from 0.1 micron to 80 microns, preferably around 20-30 microns, while the thickness of the coating film is preferably approximately 0.2 to 0.3 micron.

The internal diameter of this tubular device depends on which segment must be replaced:
in the case of urethra, this tubular device has an innner diameter that varies from 16 to 24 Ch max (1 Charrier=⅓ mm);
in the case of ureters, this tubular device has an inner diameter that varies from 6 to 10 Ch max.

The application of the coating layer in pyrolytic turbostratic carbon takes place according to a known technique, for example by means of PVD.

These patches and tubular devices made of coated silicone may also be reinforced by incorporating in the thickness a reinforcing mesh in Dacron in order to facilitate the suture to the bladder and/or urethra/ureters, avoiding the tearing of the sutures, and/or to provide a greater rigidity according to the need.

In the present method resorbable rigid scaffolds can also be used such as that described in WO2011/018300, or resorbable cuffs, such as those described in the applications MI2011A 000386, MI2012A 000380, or non-resorbable hemispheres such as those described in the application MI2011A 000387.

Further features of the invention will be made clearer by the following detailed description referred to its embodiments purely by way of a non-limiting example, illustrated in the accompanying drawings, in which:

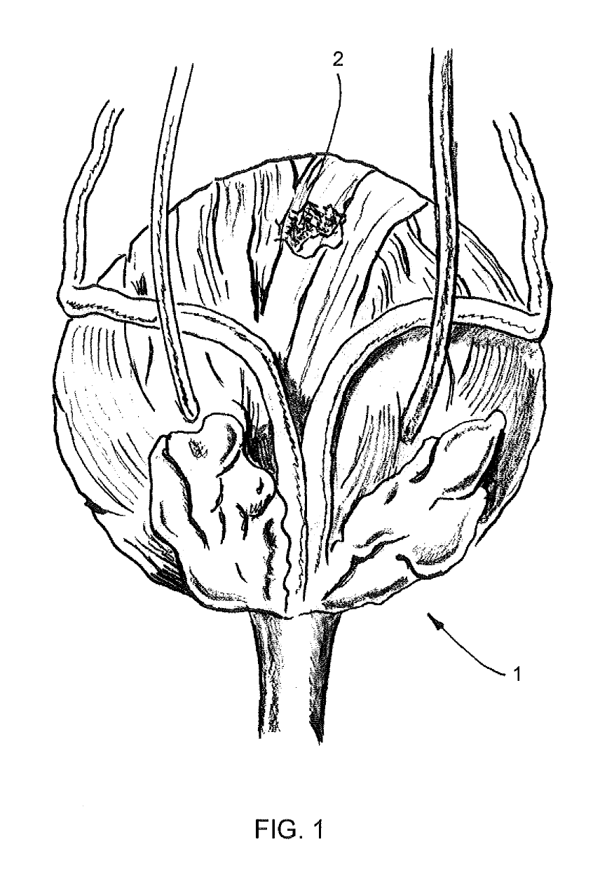

FIG. 1 is a rear frontal view of a bladder affected by neoplasia (darkened area), shown with the relative ureters and urethra partially interrupted;

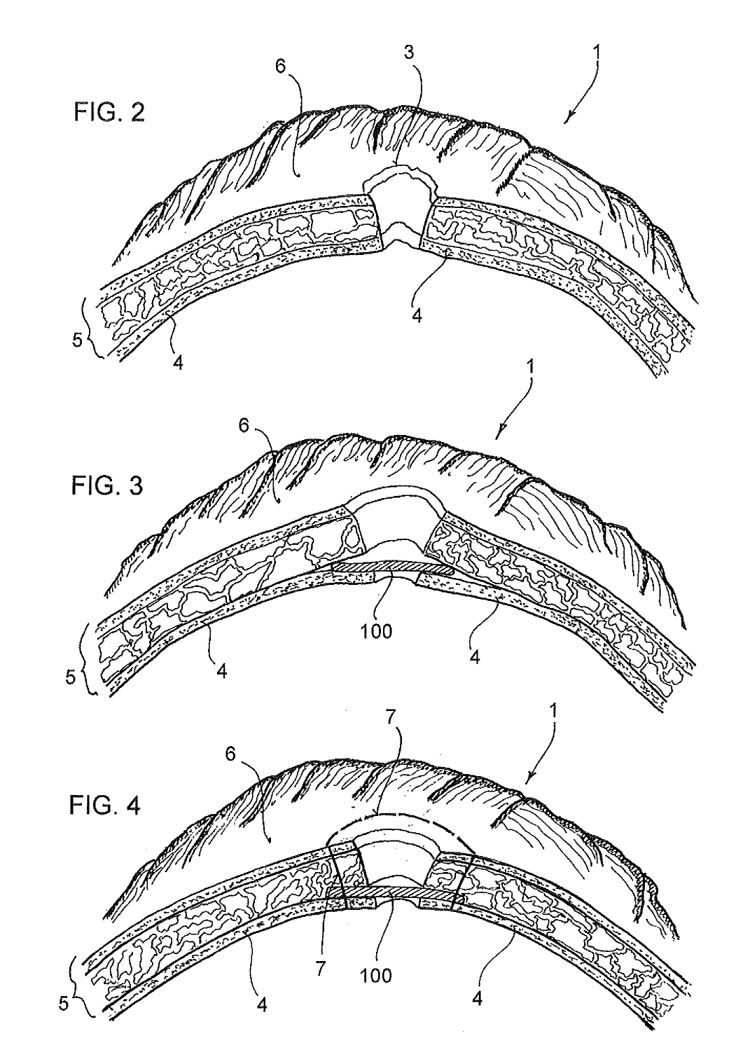

FIG. 2 is a vertical sectioned view of the bladder of FIG. 1 wherefrom the portion of bladder wall affected by neoplasia (darkened area removed) has been removed during the present method of grafting;

FIG. 3 is a vertical sectioned view of bladder similar to that of FIG. 2 illustrated after the insertion of a patch;

FIG. 4 is a vertical sectioned view of bladder similar to that of FIGS. 2 and 3 illustrated after suturing of the patch to said bladder.

A description will now be given of the method of implanting of the present invention, referring mainly to the replacement of a portion of bladder wall of a bladder 1 affected by a disease, even if this method is applicable to the urethra and/or ureters affected by specific diseases thereof.

In general, as diseases of the bladder and/or ureters/urethra, mention can be made of stenosis, neoplasias, necrosis, injuries, iatrogenic lesions and the like, and congenital malformations (low bladder capacity).

Referring to FIG. 1, in the surgical method of the present invention the first action is the removal from said bladder 1 of a portion 2 of bladder wall affected by disease.

The bladder wall, both that removed 2 and the healthy remaining one, is essentially formed by two layers of different biological tissue: an external muscle tissue 5 and an internal urothelial tissue 4, as illustrated in FIG. 2.

The removal of this diseased portion 2 leads to the formation of a hole 3 in the bladder 1 to be covered by a special patch 100 (FIG. 3).

In the art these patches are applied by resting them on the external surface 6 of the muscle tissue 5 of the bladder 1, then suturing them along their perimeter around the edge of the hole 3 in end-to-end mode: in the present method instead the process involves the performing of a first phase of detachment of a portion of the muscle tissue 5 from the underlying urothelial tissue 4 (FIG. 3) near the edge of the hole 3 in order to be able to insert the perimeter edge of the patch 100 between the detached flaps of the two muscle 5 and urothelial 4 tissues.

Subsequently the detached flaps of the two tissues are brought together to close as a sandwich the edge of said patch 100 (FIG. 4).

Following this bringing together a suture thread 7 is passed through the muscle 5 and urothelial 4 tissues that enclose the edge of said patch 100 so as to suture said patch 100 to the bladder 1 around the whole perimeter of the hole 3.

The suture thread 7 can be made of a resorbable material, for example PGA, or non-resorbable one, with diameter 3/0 or 4/0.

This technique means that the edge of the patch 100 or at least one portion of said edge is located interposed between the muscle tissue and the urothelial tissue previously detached, at the perimeter of the above said removed portion: in this position the artificial patch will behave as a support for the regrowing neotissue, both for the layer of muscle tissue 5 and the layer of urothelial tissue 4 which will go to cover respectively the external surface and the internal surface of said patch.

The same method described above is also applicable in the case wherein a cuff or hemispherical device has to be implanted for bladder augmentation.

In the case of grafting of an artificial tubular device in place of a urethral or ureteral segment an intermediate step also has to be provided, prior to the step of detachment of the muscle tissue from the underlying urothelial tissue, in which to perform a small longitudinal incision of the external wall at the free end of each segment of ureter (or urethra) which is remained after the removal of the tubular segment affected by disease.

In this way an underlying tubular portion of urothelial tissue is left uncovered and on view at each free end of each portion (remaining segment) of ureter/urethra remaining after the removal of the tubular segment affected by disease.

The aim of this incision is to cut only the layer of muscle tissue which forms the external surface of the ureter and urethra, and to create two flaps of muscle tissue so as to leave intact and on view the underlying urothelial tissue of tubular shape.

At this point, the forced insertion of the tubular portion of urothelial tissue, left uncovered, is provided inside the artificial tubular device, fitting the ends of the artificial tubular device over the corresponding free ends of the tubular urothelial tissues of the remained segments.

Subsequently the flaps of the incision of the external muscle tissue will be sutured longitudinally one to the other so as to cover each end of the artificial tubular device and restore the tubular shape of said external muscle tissue.

At this point the tubular device will be attached to both muscle and urothelial tissues, suturing it along the circumference of both its ends, by means of thread for sutures with diameter 3/0 or 4/0, in the resorbable material, for example PGA, or non-resorbable one.

The present invention is not limited to the particular embodiments described previously and illustrated in the accompanying drawings, but instead numerous detail changes may be made thereto within the reach of the person skilled in the art, without thereby departing from the scope of the same invention, as defined in the appended claims.

1. Method for implanting an artificial implant (100) provided with perimeter edge, in a bladder and/or in an urethra/ureter in the surgical treatment of diseases of the bladder and/or of the urethra/ureters by means of removal of a diseased bladder portion and/or of diseased urethra/ureters segment, characterised in that it provides a step of interposing of said edge of said artificial implant (100) between the muscle tissue (5) and the underlying urothelial tissue (4), which are previously detached at the edge of the removed bladder portion or at the free ends of the urethra/ureter portions (segments) remaining after removal of the diseased segment, so as to be sandwich sutured between said muscle tissue (5) and said urothelial tissue (4).

2. Method according to claim 1 comprising the following steps:
removing the portion of bladder and/or the segment of urethra/ureters of a patient affected by said disease;
detaching a portion of said muscle tissue (5) from the underlying portion of urothelial tissue (4) at the edge of said removed bladder portion or at the free ends of the urethra/ureter portions (segments) remaining after removal of the diseased segment, so as to form flaps of muscle tissue (5) detached from the urothelial tissue;
inserting, between the detached flaps of muscle tissue (5) and the urothelial tissue (4) of the perimeter edge, said artificial implant selected from among patch, scaffold, cuff, hemispherical device and/or tubular device;
bringing together said detached flaps until closing as a sandwich the perimeter edge of said patch, scaffold, cuff, hemispherical device and/or tubular device;
passing a suture thread through said brought together flaps of muscle tissue (5) and urothelial tissue (4) so as to suture the perimeter edge of said patch, scaffold, cuff, hemispherical device and/or tubular device to both of said tissues.

3. Method according to claim 2 wherein said implant is an absorbable (resorbable) or non-absorbable (non-resorbable) patch, preferably made with a flexible and elastic material, optionally reinforced, more preferably in soft and elastic silicone, completely coated by pyrolytic turbostratic carbon or by diamond-like carbon.

4. Method according to claim 3 wherein the thickness of the patch is around 600 microns and the thickness of the coating film is approximately 0.2 to 0.3 micron.

5. Method according to claim 1 wherein the implant is a tubular device in elastic and soft silicone coated with pyrolytic turbostratic carbon, optionally reinforced, having a thickness ranging from 0.1 micron to 80 microns, preferably around 20-30 microns, with a coating thickness preferably approximately 0.2 to 0.3 micron.

6. Method according to claim 1 wherein said implant is selected from among rigid scaffolds, preferably resorbable, resorbable cuffs, or non-resorbable hemispheres.

7. Method according to claim 1 wherein the suture is carried out with a suture thread (7) in resorbable or non-resorbable material, with diameter 3/0 or 4/0.

8. Method according to claim 1 wherein, when the implant to be grafted is an artificial tubular device, before the detachment of the muscle tissue (5) from the urothelial tissue (4), an additional step is provided comprising the longitudinal incision of the external wall of each free end of respective ureter/urethra segments remained after the removal of the diseased ureter/urethra segment, so as to form flaps of muscle tissue (5) and leave uncovered and on view a tubular portion of urothelial tissue at each free end of said urethra/ureter portions (segments) remaining after removal of the diseased segment.

9. Method according to claim 8 wherein after said detachment of the muscle tissue (5) from the urothelial tissue (4), the following steps are also provided
forced insertion, inside the artificial tubular device, of the tubular portion of the left uncovered urothelial tissue of the respective urethra/ureters segment remaining after removal of said diseased segment;
suture of said incision flaps of the muscle tissue (5) so as to cover each end of the artificial tubular device and restore the tubular shape of said external muscle tissue (5).

10. Method according to claim 2 wherein the implant is a tubular device in elastic and soft silicone coated with pyrolytic turbostratic carbon, optionally reinforced, having a thickness ranging from 0.1 micron to 80 microns, preferably around 20-30 microns, with a coating thickness preferably approximately 0.2 to 0.3 micron.

11. Method according to claim 2 wherein said implant is selected from among rigid scaffolds, preferably resorbable, resorbable cuffs, or non-resorbable hemispheres.

12. Method according to claim 2 wherein the suture is carried out with a suture thread (7) in resorbable or non-resorbable material, with diameter 3/0 or 4/0.

13. Method according to claim 2 wherein, when the implant to be grafted is an artificial tubular device, before the detachment of the muscle tissue (5) from the urothelial tissue (4), an additional step is provided comprising
the longitudinal incision of the external wall of each free end of respective ureter/urethra segments remained after the removal of the diseased ureter/urethra segment, so as to form flaps of muscle tissue (5) and leave uncovered and on view a tubular portion of urothelial tissue at each free end of said urethra/ureter portions (segments) remaining after removal of the diseased segment.
